(19) United States
(12) Patent Application Publication
(10) Pub. No.: US 2026/0318897 A1
(43) Pub. Date: Oct. 15, 2026

(54) CONTROLLING SOIL MOISTURE LEVEL FOR EARTHWORM ACTIVITY MANAGEMENT TO PREVENT RUNOFF

(71) Applicant: International Business Machines Corporation, Armonk, NY (US)

(72) Inventors: Sarbajit Kumar RAKSHIT, Kolkata (IN); Jagabondhu HAZRA, Bangalore (IN); Manikandan PADMANABAN, Chennai (IN)

(21) Appl. No.: 19/178,139

(22) Filed: Apr. 14, 2025

(51) Int. Cl. G01N 33/24; A01B 79/00; G01W 1/10; G06T 17/05; G06V 20/13

(52) U.S. Cl. CPC G01N 33/246; A01B 79/005; G01N 33/245; G01W 1/10; G06T 17/05; G06V 20/13

(57) ABSTRACT

A computer system receives input data that includes a satellite observation of a farm field, weather forecasts for the farm field, and geo-coordinates of the farm field. The computer system determines a target soil moisture level that is based on tunneling activity of earthworms in the farm field. The computer system outputs the target soil moisture level.

### BACKGROUND

This disclosure relates to water and soil management. Water bodies around the world are subject to various environmental factors that can impact their quality and ecosystems. The management of land-based activities is important for maintaining the health and balance of aquatic ecosystems.

### SUMMARY

Some aspects described herein relate to a method. The method may include receiving input data that includes a satellite observation of a farm field, weather forecasts for the farm field, and geo-coordinates of the farm field. The method may include determining a target soil moisture level that is based on tunneling activity of earthworms in the farm field. The method may include outputting the target soil moisture level.

Some aspects described herein relate to a computer system. The computer system may include a processor set, one or more computer-readable storage media, and program instructions stored on the one or more computer-readable storage media to cause the processor set to perform operations. The operations may include receiving input data comprising satellite observations, weather forecasts, farm geo-coordinates, digital elevation data, and excess soil nutrient levels associated with a farm field. The operations may include determining, based on the input data, a target soil moisture level associated with accelerating tunneling activity of earthworms while minimizing greenhouse gas emissions from the earthworms. The operations may include outputting the target soil moisture level.

Some aspects described herein relate to a computer program product. The computer program product may include one or more computer-readable storage media and program instructions stored on the one or more computer-readable storage media to perform operations. The operations may include detecting one or more waterbodies having high potential for algal bloom using a combination of satellite observations, weather forecast, farm geo-coordinates, and digital elevation information. The operations may include identifying a farm field having a high potential for contributing to water runoff with excess soil nutrients, based on the one or more waterbodies, the satellite observations, the weather forecast, and the digital elevation information. The operations may include estimating an excess soil nutrient level for the farm field. The operations may include outputting an optimal number of earthworms to deploy in the farm field based on the estimated excess soil nutrient level.

### BRIEF DESCRIPTION OF THE DRAWINGS

FIG. 1 is a diagram of an example computing environment for determining a target soil conversation level.

FIG. 2 is a diagram illustrating the process of water runoff from a farm field into a water body, leading to algal blooms.

FIG. 3A is a diagram of an example implementation associated with providing an optimal soil moisture level or an optimal earthworm deployment.

FIG. 3B is a diagram of an example computer for determining a target soil conversation level.

FIG. 4 is a diagram of an example implementation associated with controlling water runoff in a waterway by estimating the target soil moisture of a farm field.

FIG. 5 is a diagram of an example of a spatial table of soil moisture.

FIG. 6 is a flowchart of an example process associated with controlling a soil moisture level for earthworm activity management to prevent runoff.

FIG. 7 is a flowchart of an example process associated with controlling soil moisture level for earthworm activity management to prevent runoff.

FIG. 8 is a flowchart of an example process associated with controlling soil moisture level for earthworm activity management to prevent runoff.

### DETAILED DESCRIPTION

The following detailed description of example implementations refers to the accompanying drawings. The same reference numbers in different drawings may identify the same or similar elements.

Excessive growth of algae in water bodies, also known as algal blooms, can have severe environmental and health impacts. Such blooms are often triggered by an abundance of nutrients in the water, which can originate from agricultural runoff, including fertilizers containing phosphorus and other nutrients. These nutrients can be washed off farm fields and into nearby water bodies, contributing to the growth of algal blooms.

The current methods of controlling algal blooms focus primarily on reducing the amount of nutrients in the water. However, these approaches have limitations and do not always address the root cause of the problem. A major issue with existing methods is their inability to manage excess nutrient content in the soil effectively, which leads to the runoff of these nutrients into water bodies. As a result, algal blooms continue to form, and efforts to control them remain inadequate.

Some implementations described herein provide a computer system that determines a soil moisture level for a farm field that is related to earthworm tunnel activity. Earthworm tunneling helps the ground to absorb water rather than have the water all run along the surface. Earthworms can create multiple tunnels or burrows in a day, depending on the availability of food, moisture, and other factors. When soil moisture levels are high, earthworms are more active as high moisture levels make it easier for earthworms to move through the soil and find food. Earthworm tunnels can be used to store nutrient rich soil and soil water. This reduces the nutrient content on run-off water and prevents algal bloom to grow.

For example, the computer system may receive input data that includes a satellite observation of the farm field, weather forecasts for the farm field, and geo-coordinates of the farm field. The computer system may determine and output a target soil moisture level that is based on tunneling activity of earthworms in the farm field. The computer system may also estimate an excess soil nutrient level in the farm field based on the satellite observation, and predict rainfall and temperature patterns that affect the tunneling activity of the earthworms based on the weather forecasts. The computer system may determine areas of the farm field with high potential for water runoff based on digital elevation data and the geo-coordinates. The computer system may also determine and output an optimal number of earthworms to deploy to the farm field.

In this way, the computer system may optimize soil moisture levels to maximize earthworm tunneling activity, thereby reducing excess soil nutrient runoff into nearby water bodies and moderating algal bloom growth. The computer system may also optimize soil aeration and carbon-to-nitrogen ratios to minimize earthworm greenhouse gas emissions. Additionally, the computer system may optimize crop water and nutrient allocation, minimizing waste and runoff. As a result, the system may conserve water resources, reduce nitrogen and phosphorus pollution in water bodies, and promote efficient use of agricultural inputs.

By addressing the root cause of algal blooms, the computer system may reduce the computational resources, memory resources, and network resources required for traditional methods of controlling algal blooms, such as monitoring and forecasting algal bloom growth, and treating symptoms of the problem. The computer system may also reduce the processing resources, memory resources, and network resources required for mitigating the economic and environmental impacts of algal blooms, such as predicting and responding to algal bloom events, and assessing environmental damage. In this way, the system may conserve processing resources, memory resources, network resources, and/or the like, while optimizing farm field operations and promoting environmental sustainability.

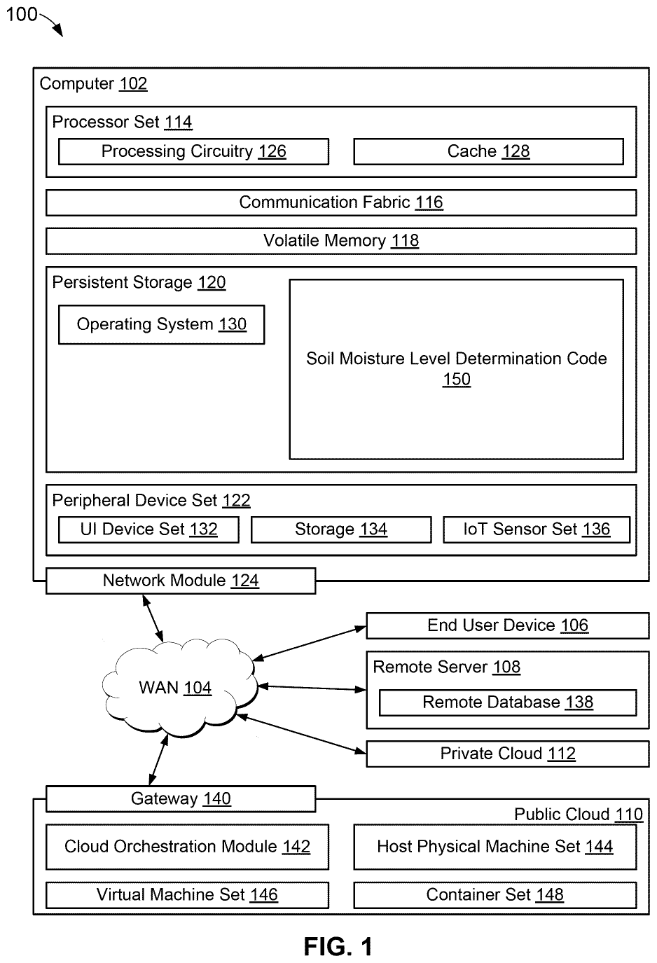

FIG. 1 is a diagram of an example computing environment 100 for determining a target soil conversation level as described herein.

Computing environment 100 contains an example of an environment for the execution of at least some of the computer code involved in performing the inventive methods, such as soil moisture level determination code 150. In addition to soil moisture level determination code 150, computing environment 100 includes, for example, computer 102, wide area network (WAN) 104, end user device (EUD) 106, remote server 108, public cloud 110, and private cloud 112. In this embodiment, computer 102 includes processor set 114 (including processing circuitry 126 and cache 128), communication fabric 116, volatile memory 118, persistent storage 120 (including operating system 130 and soil moisture level determination code 150, as identified above), peripheral device set 122 (including user interface (UI) device set 132, storage 134, and Internet of Things (IoT) sensor set 136), and network module 124. Remote server 108 includes remote database 138. Public cloud 110 includes gateway 140, cloud orchestration module 142, host physical machine set 144, virtual machine set 146, and container set 148.

Computer 102 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network, or querying a database, such as remote database 138. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 102, to keep the presentation as simple as possible. Computer 102 may be located in a cloud, even though it is not shown in a cloud in FIG. 1. On the other hand, computer 102 is not required to be in a cloud except to any extent as may be affirmatively indicated.

Processor set 114 includes one, or more, computer processors of any type now known or to be developed in the future. Processing circuitry 126 may be distributed over multiple packages (for example, multiple, coordinated integrated circuit chips). Processing circuitry 126 may implement multiple processor threads and/or multiple processor cores. Cache 128 is memory that is located in the processor chip package(s) and is typically used for data or code that should be available for rapid access by the threads or cores running on processor set 114. Cache memories are typically organized into multiple levels depending upon relative proximity to the processing circuitry. Alternatively, some, or all, of the cache for the processor set may be located “off chip.” In some computing environments, processor set 114 may be designed for working with qubits and performing quantum computing.

Computer-readable program instructions are typically loaded onto computer 102 to cause a series of operational steps to be performed by processor set 114 of computer 102 and thereby effect a computer-implemented method, such that the instructions thus executed will instantiate the methods specified in flowcharts and/or narrative descriptions of computer-implemented methods included in this document (collectively referred to as “the inventive methods”). These computer-readable program instructions are stored in various types of computer-readable storage media, such as cache 128 and the other storage media discussed below. The program instructions, and associated data, are accessed by processor set 114 to control and direct performance of the inventive methods. In computing environment 100, at least some of the instructions for performing the inventive methods may be stored in soil moisture level determination code 150 in persistent storage 120.

Communication fabric 116 is the signal conduction path that allows the various components of computer 102 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

Volatile memory 118 is any type of volatile memory now known or to be developed in the future. Examples include dynamic type random access memory (RAM) or static type RAM. Typically, volatile memory 118 is characterized by random access, but this is not required unless affirmatively indicated. In computer 102, the volatile memory 118 is located in a single package and is internal to computer 102, but, alternatively or additionally, the volatile memory may be distributed over multiple packages and/or located externally with respect to computer 102.

Persistent storage 120 is any form of non-volatile storage for computers that is now known or to be developed in the future. The non-volatility of this storage means that the stored data is maintained regardless of whether power is being supplied to computer 102 and/or directly to persistent storage 120. Persistent storage 120 may be a read only memory (ROM), but typically at least a portion of the persistent storage allows writing of data, deletion of data, and re-writing of data. Some familiar forms of persistent storage include magnetic disks and solid-state storage devices. Operating system 130 may take any of several forms, such as various known proprietary operating systems or open source Portable Operating System Interface-type operating systems that employ a kernel.

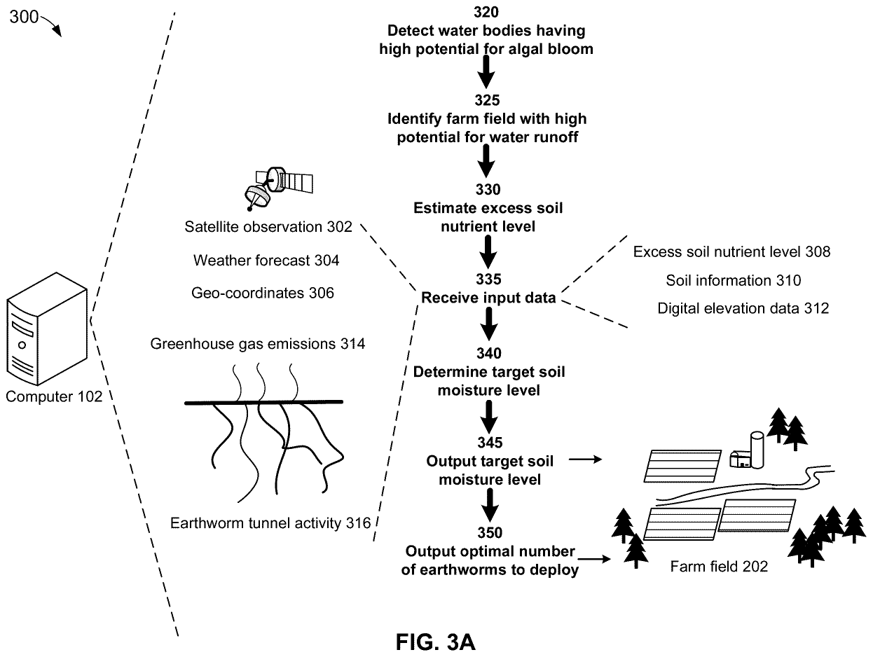

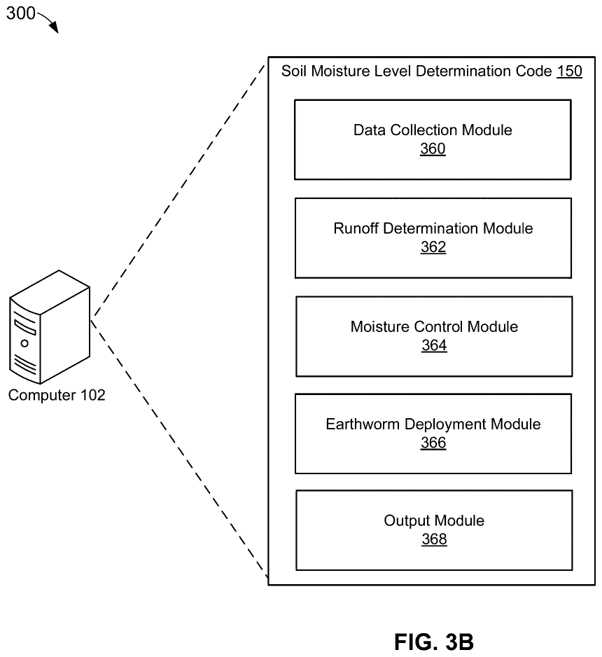

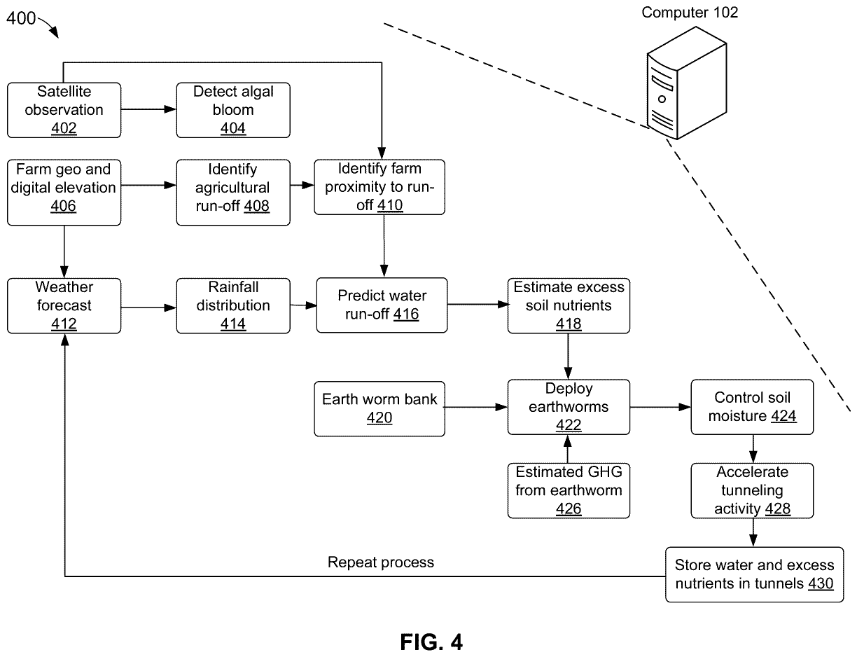

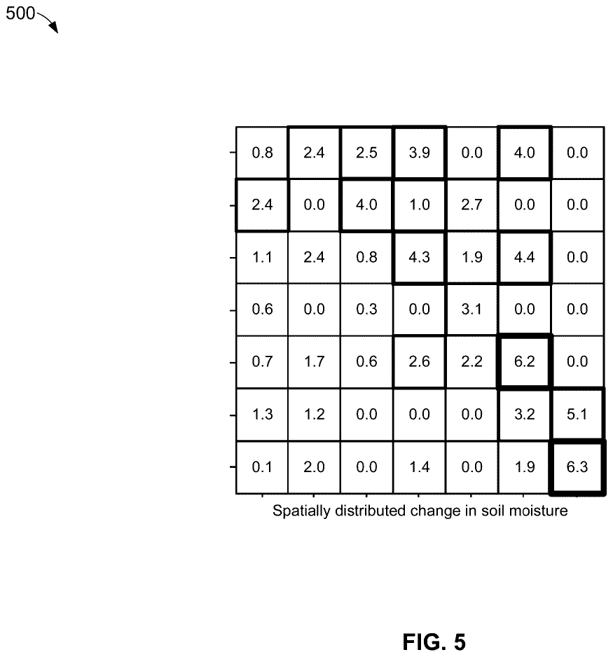

The code included in the soil moisture level determination code 150 typically includes at least some of the computer code involved in performing one or more operations described herein, such as the operations of implementation 300 in FIG. 3A, implementation 400 in FIG. 4, and the processes described in FIGS. 5-7.

Peripheral device set 122 includes the set of peripheral devices of computer 102. Data communication connections between the peripheral devices and the other components of computer 102 may be implemented in various ways, such as Bluetooth® connections, Near-Field Communication (NFC) connections, connections made by cables (such as universal serial bus (USB) type cables), insertion-type connections (for example, secure digital (SD) card), connections made through local area communication networks and/or connections made through wide area networks such as the internet. In various embodiments, UI device set 132 may include components such as a display screen, speaker, microphone, wearable devices (such as goggles and smart watches), keyboard, mouse, printer, touchpad, game controllers, and/or haptic devices. Storage 134 is external storage, such as an external hard drive, or insertable storage, such as an SD card. Storage 134 may be persistent and/or volatile. In some embodiments, storage 134 may take the form of a quantum computing storage device for storing data in the form of qubits. In embodiments where computer 102 is required to have a large amount of storage (for example, where computer 102 locally stores and manages a large database), this storage may be provided by peripheral storage devices designed for storing very large amounts of data, such as a storage area network (SAN) that is shared by multiple, geographically distributed computers. IoT sensor set 136 is made up of sensors that can be used in Internet of Things applications. For example, one sensor may be a thermometer and another sensor may be a motion detector.

Network module 124 is the collection of computer software, hardware, and firmware that allows computer 102 to communicate with other computers through WAN 104. Network module 124 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 124 are performed on the same physical hardware device. In other embodiments (for example, embodiments that utilize software-defined networking (SDN)), the control functions and the forwarding functions of network module 124 are performed on physically separate devices, such that the control functions manage several different network hardware devices. Computer-readable program instructions for performing the inventive methods can typically be downloaded to computer 102 from an external computer or external storage device through a network adapter card or network interface included in network module 124.

WAN 104 is any wide area network (for example, the internet) capable of communicating computer data over non-local distances by any technology for communicating computer data, now known or to be developed in the future. In some embodiments, the WAN 104 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and/or edge servers.

EUD 106 is any computer system that is used and controlled by an end user (for example, a customer of an enterprise that operates computer 102), and may take any of the forms discussed above in connection with computer 102. EUD 106 typically receives helpful and useful data from the operations of computer 102. For example, in a hypothetical case where computer 102 is designed to provide a recommendation to an end user, this recommendation would typically be communicated from network module 124 of computer 102 through WAN 104 to EUD 106. In this way, EUD 106 can display, or otherwise present, the recommendation to an end user. In some embodiments, EUD 106 may be a client device, such as thin client, heavy client, mainframe computer, desktop computer and so on.

Remote server 108 is any computer system that serves at least some data and/or functionality to computer 102. Remote server 108 may be controlled and used by the same entity that operates computer 102. Remote server 108 represents the machine(s) that collect and store helpful and useful data for use by other computers, such as computer 102. For example, in a hypothetical case where computer 102 is designed and programmed to provide a recommendation based on historical data, this historical data may be provided to computer 102 from remote database 138 of remote server 108.

Public cloud 110 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 110 is performed by the computer hardware and/or software of cloud orchestration module 142. The computing resources provided by public cloud 110 are typically implemented by virtual computing environments that run on various computers making up the computers of host physical machine set 144, which is the universe of physical computers in and/or available to public cloud 110. The virtual computing environments (VCEs) typically take the form of virtual machines from virtual machine set 146 and/or containers from container set 148. These VCEs may be stored as images and may be transferred among and between the various physical machine hosts, either as images or after instantiation of the VCE. Cloud orchestration module 142 manages the transfer and storage of images, deploys new instantiations of VCEs, and manages active instantiations of VCE deployments. Gateway 140 is the collection of computer software, hardware, and firmware that allows public cloud 110 to communicate through WAN 104.

Some further explanation of VCEs will now be provided. VCEs can be stored as “images.” A new active instance of a VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 112 is similar to public cloud 110, except that the computing resources are only available for use by a single enterprise. While private cloud 112 is depicted as being in communication with WAN 104, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this example, public cloud 110 and private cloud 112 are both part of a larger hybrid cloud.

Cloud computing services and/or microservices (not separately shown in FIG. 1): private and public clouds 110 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to an “as a service” technology paradigm where content is being presented to an internal or external customer in the form of a cloud computing service. As-a-service offerings typically provide endpoints with which various customers interface. These endpoints are typically based on a set of application programming interfaces (APIs). One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with such tasks. Another category is Software-as-a-Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

In some implementations, a device (e.g., computer 102, computer system) may receive input data that includes a satellite observation of a farm field, weather forecasts for the farm field, and geo-coordinates of the farm field. The device may determine a target soil moisture level that is based on tunneling activity of earthworms in the farm field. The device may output the target soil moisture level.

In some implementations, the device may receive input data comprising satellite observations, weather forecasts, farm geo-coordinates, digital elevation data, and excess soil nutrient levels associated with a farm field. The device may determine, based on the input data, a target soil moisture level associated with accelerating tunneling activity of earthworms while minimizing greenhouse gas emissions from the earthworms. The device may output the target soil moisture level.

In some implementations, the device may detect one or more waterbodies having high potential for algal bloom using a combination of satellite observations, weather forecast, farm geo-coordinates, and digital elevation information. The device may identify a farm field having a high potential for contributing to water runoff with excess soil nutrients, based on the one or more waterbodies, the satellite observations, the weather forecast, and the digital elevation information. The device may estimate an excess soil nutrient level for the farm field. The device may output an optimal number of earthworms to deploy in the farm field based on the estimated excess soil nutrient level.

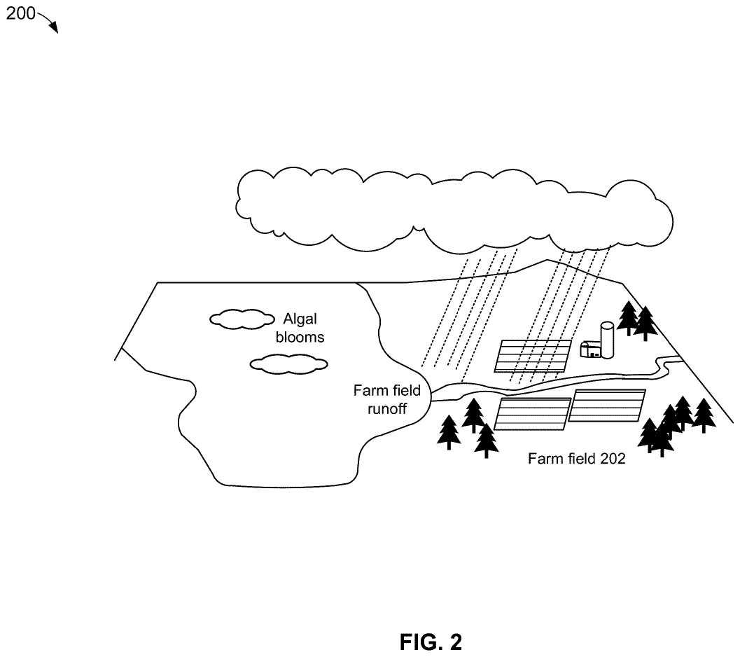

FIG. 2 is a diagram illustrating an example 200 of the process of water runoff from a farm field 202 into a waterbody, leading to algal blooms. As shown, the farm field 202 is receiving rain, causing excess rain water to run off the surface of the farm field 202 into a water body. The water body is depicted as a lake in FIG. 2, but the lake may also represent other types of water bodies, such as rivers, reservoirs, estuaries, or oceans.

As the excess surface water from the farm field 202 flows into the water body, the water carries with it excess nutrients and sediments, such as nitrogen, phosphorus, and soil particles. These excess nutrients and sediments can stimulate the growth of algae, leading to the formation of algal blooms. The algal blooms may be viewed as large, greenish-colored patches on the surface of the water body. These blooms can be composed of various types of algae, including cyanobacteria, which can produce toxins that are harmful to humans, animals, and aquatic life.

The process illustrated in FIG. 2 is a common problem in many agricultural regions, where excess nutrients and sediments from farm fields can enter water bodies and cause algal blooms. These blooms can have significant environmental and economic impacts, including degrading water quality, harming aquatic ecosystems, and affecting human health.

In some implementations, a computer system (e.g., computer 102) may mitigate this problem by optimizing a soil moisture level of the farm field 202 for earthworm tunneling activity to increase the soil absorption of the water and reduce the excess nutrient runoff from farm field 202. By using satellite data, weather forecasts, and other information, the computer system can predict the target (optimal) soil moisture level and the optimal number of earthworms that would prevent excess water runoff and associated algal blooms. This leads to improved water quality, reduced environmental impacts, and enhanced agricultural productivity.

FIG. 3A is a diagram of an example implementation 300 associated with providing an optimal soil moisture level or an optimal earthworm deployment. As shown in FIG. 3A, example implementation 300 includes a computer system (e.g., computer 102). FIG. 3B illustrates components of the computer system, such as the soil moisture level determination code 150 of FIG. 1 that may perform operations of a computer-implemented method shown in FIG. 3A. The soil moisture level determination code 150 may include a data collection module 360, a runoff determination module 362, a moisture control module 364, an earthworm deployment module 366, and an output module 368. Each module of the soil moisture level determination code 150 may operate using the processor set 114, communication fabric 116, volatile memory 118, and persistent storage 120 of computer 102 illustrated in FIG. 1.

By processing the input data to generate the instructions for controlling soil moisture, addressing the root cause of algal blooms, the modules of the computer system may reduce the computational resources, memory resources, and network resources required for traditional methods of controlling algal blooms, such as monitoring and forecasting algal bloom growth, and treating symptoms of the problem.

As shown by reference number 320 of FIG. 3A, the computer system (e.g., using a data collection module 360) may collect different type of data. For example, the computer system may detect water bodies with a high potential for algal blooms (e.g., compared to a threshold, top quartile, or percentage) as compared to an average propensity for algal blooms in water bodies generally. The computer system may receive input data about historical bloom information (e.g., temperature history, rain history) for water bodies based on certain conditions. The computer system may also receive data about such current conditions (e.g., temperature, rain, snow melt). The computer system may receive satellite observation data 302, a weather forecast 304, and geo-coordinates 306 of water bodies and farm fields that may factor into such determinations.

As shown by reference number 325, the computer system (e.g., using a runoff determination module 362) may identify a culturable field with a high potential for water runoff containing excess soil nutrients, such as farm field 202. As shown by reference number 330, the computer system (e.g., using a moisture control module 364) may also estimate an excess soil nutrient level 308 for the farm field 202 based on soil information 310 (e.g., soil type, soil texture, soil organic matter content, soil composition). For example, the computer system may estimate soil nutrient levels through microwave/multi-spectral satellite imageries. As shown by reference number 335, the computer system (e.g., using the data collection module 360) may receive any combination of this information as input data. Input data may also include digital elevation data 312 about the surface of an area that indicates how water would flow.

As shown by reference number 340, the computer system (e.g., using a moisture control module 364) may determine a target soil moisture level for the farm field 202 based on the input data. For example, the system may receive the input data through a digital interface or a communication interface to a satellite receiver and a weather forecasting system. The target soil moisture level may be an optimum moisture level for earthworm tunneling activity 316 (e.g., not too much activity, or not too little activity). For example, if the satellite observation data indicates a particular nutrient composition and a weather forecast of no rain followed by a lot of rain, the computer system may indicate that some watering is to take place to spur earthworm activity over the next several days in advance of a possible weather development calling for heavy rain. The computer system may predict rainfall and temperature patterns affecting earthworm tunnel activity. The computer system may take into account satellite observations of current algal blooms or conditions for algal blooms. The computer system may determine the target moisture level based on maintaining adherence to a soil nutrient requirement and/or a water usage constraint.

As shown by reference number 345, the computer system (e.g., using an output module 368) may output an indication of this target soil moisture level. The output may include a report for how much moisture (e.g., water time per hectare, etc.) the operator of the farm field 202 would add, not add, or be aware of to maximize earthworm activity to maximize surface water absorption. The earthworm tunnel activity may take place on or off of cultivated crop land. The computer system may generate recommendations for irrigation schedules and fertilizer application rates.

As shown by reference number 350, the computer system (e.g., using an earthworm deployment module 366) may output an optimal number of earthworms to deploy. This may take into account earthworm population information for the farm field 202 or the geographical area. The computer system may also take into account an amount of tunneling activity for a population of earthworms from collected data of such activity at the farm field 202 or in other locations. The computer system may determine the optimal number based on the target soil moisture level. For example, the computer system may use a model of earthworm behavior (e.g., tunnel dynamics) and soil nutrient dynamics to optimize soil moisture levels for each spatial pixel of a spatial map of the farm field 202, reducing green house gas (GHG) emissions 314 while maintaining optimal crop growth. For example, the computer may use a model of earthworm tunnel dynamics to optimize the storage of nutrient-rich soil and soil water, reducing nutrient content in run-off water and preventing algal bloom growth.

In some implementations, the computer system may consider a trade-off between additional GHG from the earthworms and the preservation of a soil nutrient. For example, the computer may use a cost-benefit analysis to weigh the benefits of deploying earthworms against the potential increase in GHG emissions 314 from their cast and adjusting the target soil moisture level to minimize emissions while maintaining optimal soil nutrient levels. The computer system may analyze the relationship between the number of earthworms, the soil moisture level, and tunnel creation.

In some implementations, the computer system may use geospatial analysis to identify areas where water is likely to overflow from waterbodies and adjust the target soil moisture level to reduce the risk of water pollution. The computer system may use historical flood data and geographic elevation to identify water overflow. For example, the computer system may use historical flood data and geographic elevation data to identify water flow issues. The computer system may analyze the historical flood data and geographic elevation to identify how water will overflow and mix with downstream water.

FIG. 4 is a diagram of an example implementation 400 associated with controlling water runoff in a waterway by estimating the target soil moisture of a farm field.

Example implementation 400 indicates various information that may contribute to the input data for a computer system (e.g., computer 102), including satellite observations 402, farm geo-coordinates and digital elevation 406, rainfall distribution 414, and a weather forecast 412. Operations that can contribute to the input data may include detecting algal bloom 404, identifying agricultural run-off 408, and identifying farms in proximity to run-off 410.

As shown by reference number 416, the computer system may predict whether there will be run-off for the farm field 202 that will be an issue. If there will be runoff, the computer system may estimate excess soil nutrients 418. The computer system may use this estimate and the input data to determine whether to recommend a number of earthworms to deploy 422 from an earth worm bank 420. The recommendation may be based on an estimated GHG emission from the earthworms 426. The computer system may also recommend a target moisture level to maintain, as part of controlling the soil moisture 424. The target soil moisture level, if calculated properly, may accelerate tunneling activity 428 to store water with excess nutrients in the tunnels 430.

The target moisture level may be based in part on the weather forecast 412. For example, the computer system may delay or adjust a recommended addition of moisture to the soil based on when rain is expected and how much rain is expected.

The computer system may analyze the nutrient requirements of the crops being grown in the farm field and adjust the target soil moisture level accordingly to ensure optimal crop growth while minimizing water runoff. The computer system may also analyze the water stress tolerance of the crops being grown in the farm field and adjust the target soil moisture level accordingly to prevent water stress while minimizing water runoff to mitigate or prevent algal blooms in nearby or downstream water bodies. After water and excess nutrients are stored in the earthworm tunnels, the computer system may repeat this process for the next iteration of evaluating actions to mitigate or prevent runoff.

FIG. 5 is a diagram of an example 500 of a spatial table of soil moisture, highlighting the importance of controlling soil moisture levels to prevent algal blooms. Example 500 shows a spatial table with different soil moisture levels, with higher soil moisture levels indicating a greater potential for algal blooms. The spatial table may also indicate changes in soil moisture. Each square (pixel) in the spatial table is a different spatial location of the farm field 202. Different regions have different soil moisture levels and corresponding changes in soil moisture.

Soil borrowing refers to the activity of earthworms absorbing and processing soil nutrients through their tunneling and cast (i.e., waste) production. Earthworms play a crucial role in soil ecosystems, and the tunneling activity helps to aerate the soil, improve soil structure, and facilitate the movement of water and nutrients. Given a spatially distributed farm location of interest, nutrient holding activity is accelerated by choosing optimal soil moisture for each spatial pixel such that the soil borrowing activity is increased for increased absorption of soil nutrients.

The borrowing activity and moisture levels may have different constraints and functions. For example, the computer system may calculate a target moisture level and/or earthworm deployment number based on a cost function J that considers a reduction in the overall GHG emission produced by the earthworm cast/guts without compromising on the crop water stress, nutrient requirements, and the earthworm's survival soil moisture and temperature. The computer system may use the following equations in such calculations.

The soil borrowing to store soil nutrients and the GHG emission may be represented as a cost function J=Max.

$${\sum }_{{j = 1}_{t}}^{N}$$

Soil_borrow(SM jt, Tjt)_{j,t}−GHG(SM jt, Tjt)_{j,t}), where Soil_borrow (SM jt, Tjt)_{j,t}represents the soil nutrients absorbed through soil borrowing activity which is a function of soil moisture (SM) and soil temperature (T) at pixel j for time t, and GHG(SM, T)_{j,t}represents the total GHG emission emitted by earthworm cast/gut activity which is a function of SM and T at pixel j for time t. The different elements of the constraints may include Cast (SM, T)j,t (earthworm cast production for pixel j at time t), C_Nutrients_{j,t} (crop nutrients requirements for at pixel j at time t), C_{water j,t}(crop water requirements at pixel j for a time t), SM_{min,j,t}, SM_{max,j,t} (minimum and maximum soil moisture for earthworm's survival), andT_{min,j,t}, T_{max,j,t} (minimum and maximum temperature for earthworm's survival). The crop nutrient requirements constraint may be represented asCas (SM jt, Tjt)_{j,t}≥C_Nutrients_{j,t}. The crop water stress constraint may be represented as C_water_{min,j,t}≥C_{water j,t}(SMjt)≤C_water_{max,j,t}. The earthworm survival constraint may be represented as SM_{min,j,t}≥SM_{j,t}≤SM_{max,j,t} and ; T_{min,j,t}≥T(SM j)_{j,t}≤T_{max,j,t}.

Using the above equations, the optimal soil moisture level and earthworm deployment strategy can be determined for each pixel j at time t. For instance, for pixel 1 at time 1, the optimal soil moisture level might be 0.45, and the optimal number of earthworms to deploy might be 500. Similarly, for pixel 2 at time 2, the optimal soil moisture level might be 0.55, and the optimal number of earthworms to deploy might be 600. By using these calculations, the computer system may also help to not waste water or earthworm resources.

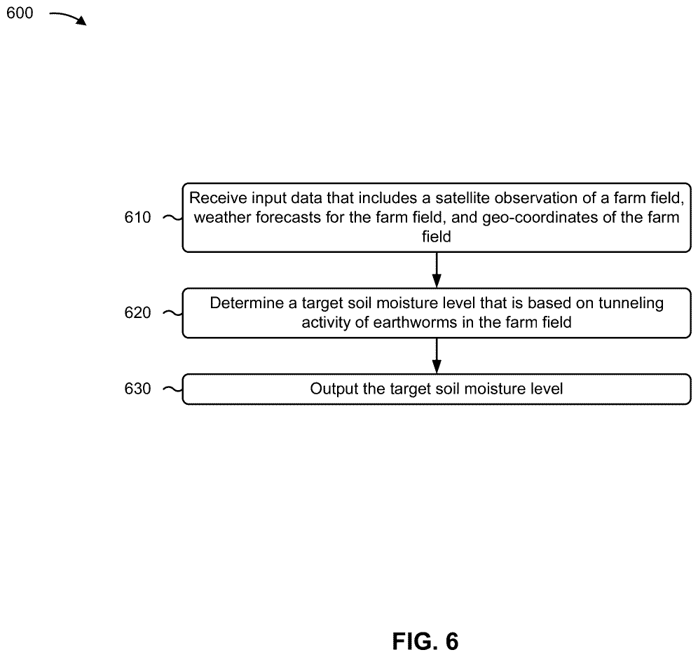

FIG. 6 is a flowchart of an example process 600 associated with controlling a soil moisture level for earthworm activity management to prevent runoff. One or more process blocks of FIG. 6 are performed by a computer system (e.g., computer 102) and/or by another device or a group of devices separate from or including the computer system.

As shown in FIG. 6, process 600 includes receiving input data that includes a satellite observation of a farm field, weather forecasts for the farm field, and geo-coordinates of the farm field (block 610). For example, the computer system may receive input data that includes a satellite observation of a farm field, weather forecasts for the farm field, and geo-coordinates of the farm field, as described above.

As further shown in FIG. 6, process 600 includes determining a target soil moisture level that is based on tunneling activity of earthworms in the farm field (block 620). For example, the computer system may determine a target soil moisture level that is based on tunneling activity of earthworms in the farm field, as described above.

As further shown in FIG. 6, process 600 includes outputting the target soil moisture level (block 630). For example, the computer system may output the target soil moisture level, as described above.

Process 600 may include additional implementations, such as any single implementation or any combination of implementations described below and/or in connection with one or more other processes described elsewhere herein.

In a first implementation, the input data includes an excess soil nutrient level in the farm field.

In a second implementation, alone or in combination with the first implementation, process 600 includes estimating an excess soil nutrient level in the farm field based on the satellite observation, where the determining of the target soil moisture level comprises determining the target soil moisture level based on the excess soil nutrient level.

In a third implementation, alone or in combination with one or more of the first and second implementations, process 600 includes predicting rainfall and temperature patterns that affect the tunneling activity of the earthworms based on the weather forecasts, where the determining of the target soil moisture level comprises determining the target soil moisture level based on the rainfall and temperature patterns.

In a fourth implementation, alone or in combination with one or more of the first through third implementations, the input data includes digital elevation data for the farm field, and process 600 includes determining areas of the farm field with high potential for water runoff based on the digital elevation data and the geocoordinates, and the determining of the target soil moisture level comprises determining the target soil moisture level based on the areas with high potential for water runoff.

In a fifth implementation, alone or in combination with one or more of the first through fourth implementations, the determining of the target soil moisture level comprises determining the target soil moisture level based on a crop nutrient requirement and a crop water stress constraint.

In a sixth implementation, alone or in combination with one or more of the first through fifth implementations, the determining of the target soil moisture level comprises determining the target soil moisture level based on minimizing greenhouse gas emissions from the earthworms.

In a seventh implementation, alone or in combination with one or more of the first through sixth implementations, the determining of the target soil moisture comprises determining the target soil moisture using a cost function that is based on the tunneling activity of the earthworms, the greenhouse gas emissions from the earthworms, and crop nutrient requirements.

In an eighth implementation, alone or in combination with one or more of the first through seventh implementations, the determining of the target soil moisture level comprises determining the target soil moisture level based on a soil type, a soil texture, and a soil organic matter content of the farm field.

Although FIG. 6 shows example blocks of process 600, in some implementations, process 600 includes additional blocks, fewer blocks, different blocks, or differently arranged blocks than those depicted in FIG. 6. Additionally, or alternatively, two or more of the blocks of process 600 may be performed in parallel.

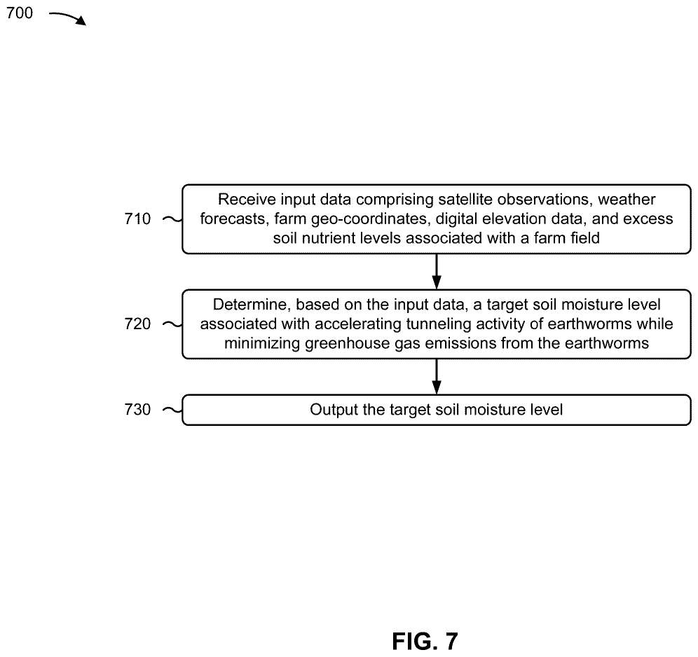

FIG. 7 is a flowchart of an example process 700 associated with controlling soil moisture level for earthworm activity management to prevent runoff. One or more process blocks of FIG. 7 are performed by a computer system (e.g., computer 102) and/or by another device or a group of devices separate from or including the computer system.

As shown in FIG. 7, process 700 includes receiving input data comprising satellite observations, weather forecasts, farm geo-coordinates, digital elevation data, and excess soil nutrient levels associated with a farm field (block 710). For example, the computer system may receive input data comprising satellite observations, weather forecasts, farm geo-coordinates, digital elevation data, and excess soil nutrient levels associated with a farm field, as described above.

As further shown in FIG. 7, process 700 includes determining, based on the input data, a target soil moisture level associated with accelerating tunneling activity of earthworms while minimizing greenhouse gas emissions from the earthworms (block 720). For example, the computer system may determine, based on the input data, a target soil moisture level associated with accelerating tunneling activity of earthworms while minimizing greenhouse gas emissions from the earthworms, as described above.

As further shown in FIG. 7, process 700 includes outputting the target soil moisture level (block 730). For example, the computer system may output the target soil moisture level, as described above.

Process 700 may include additional implementations, such as any single implementation or any combination of implementations described below and/or in connection with one or more other processes described elsewhere herein.

In a first implementation, process 700 includes identifying waterbodies with high potential for algal bloom and agricultural runoff based on the satellite observations and image analysis.

In a second implementation, alone or in combination with the first implementation, the outputting of the target soil moisture level comprises generating guidance information associated with an optimal soil moisture level for the target soil moisture level and an earthworm deployment strategy to prevent nutrient runoff and control algal blooms.

In a third implementation, alone or in combination with one or more of the first and second implementations, the operations further comprise determining a proximity of the farm field and one or more surrounding farm fields to one or more waterbodies, and determining how much the farm field and the surrounding farm fields contribute to runoff water with excess soil nutrients based on the proximity and a digital elevation of the farm field.

In a fourth implementation, alone or in combination with one or more of the first through third implementations, the determining of the target soil moisture level comprises determining the target soil moisture level based on a soil type, a soil texture, and a soil organic matter content of the farm field.

Although FIG. 7 shows example blocks of process 700, in some implementations, process 700 includes additional blocks, fewer blocks, different blocks, or differently arranged blocks than those depicted in FIG. 7. Additionally, or alternatively, two or more of the blocks of process 700 may be performed in parallel.

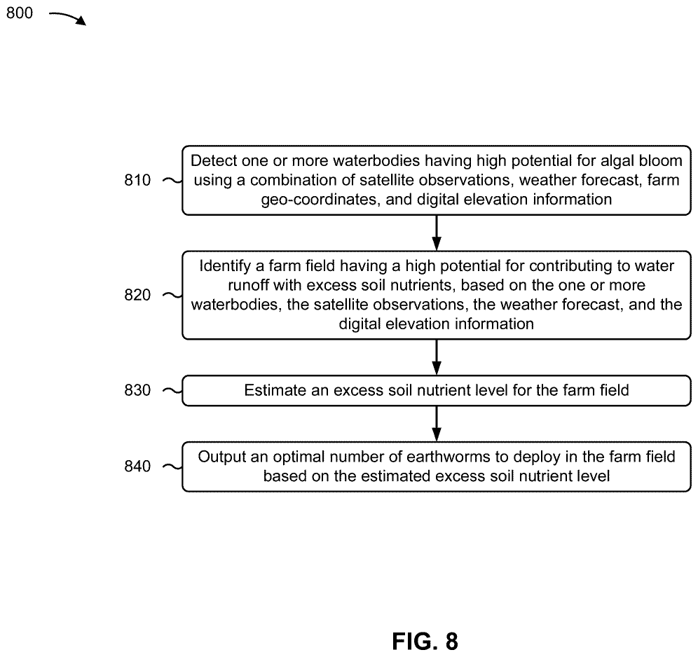

FIG. 8 is a flowchart of an example process 800 associated with controlling soil moisture level for earthworm activity management to prevent runoff. One or more process blocks of FIG. 8 are performed by a computer system (e.g., computer 102) and/or by another device or a group of devices separate from or including the computer system.

As shown in FIG. 8, process 800 includes detecting one or more waterbodies having high potential for algal bloom using a combination of satellite observations, weather forecast, farm geo-coordinates, and digital elevation information (block 810). For example, the computer system may detect one or more waterbodies having high potential for algal bloom using a combination of satellite observations, weather forecast, farm geo-coordinates, and digital elevation information, as described above.

As further shown in FIG. 8, process 800 includes identifying a farm field having a high potential for contributing to water runoff with excess soil nutrients, based on the one or more waterbodies, the satellite observations, the weather forecast, and the digital elevation information (block 820). For example, the computer system may identify a farm field having a high potential for contributing to water runoff with excess soil nutrients, based on the one or more waterbodies, the satellite observations, the weather forecast, and the digital elevation information, as described above.

As further shown in FIG. 8, process 800 includes estimating an excess soil nutrient level for the farm field (block 830). For example, the computer system may estimate an excess soil nutrient level for the farm field, as described above.

As further shown in FIG. 8, process 800 includes outputting an optimal number of earthworms to deploy in the farm field based on the estimated excess soil nutrient level (block 840). For example, the computer system may output an optimal number of earthworms to deploy in the farm field based on the estimated excess soil nutrient level, as described above.

Process 800 may include additional implementations, such as any single implementation or any combination of implementations described below and/or in connection with one or more other processes described elsewhere herein.

In a first implementation, the instructions further comprise outputting a target soil moisture level for the farm field based on the excess soil nutrient level, the one or more waterbodies, the satellite observations, the weather forecast, and the digital elevation information.

In a second implementation, alone or in combination with the first implementation, the instructions further comprise predicting, using a machine learning model, an optimal target soil moisture level based on historical data and real-time sensor data.

In a third implementation, alone or in combination with one or more of the first and second implementations, process 800 includes determining a target soil moisture level based on the estimated excess soil nutrient level, a soil type, a soil texture, and a soil organic matter content of the farm field.

In a fourth implementation, alone or in combination with one or more of the first through third implementations, the outputting of the number of earthworms comprises outputting the number of earthworms for the farm field based on an estimate of greenhouse gas emissions emitted by the number of earthworms to deploy and existing earthworms in the farm field.

In a fifth implementation, alone or in combination with one or more of the first through fourth implementations, process 800 includes outputting a target soil moisture level that is based on earthworm survival constraints.

Although FIG. 8 shows example blocks of process 800, in some implementations, process 800 includes additional blocks, fewer blocks, different blocks, or differently arranged blocks than those depicted in FIG. 8. Additionally, or alternatively, two or more of the blocks of process 800 may be performed in parallel.

The foregoing disclosure provides illustration and description, but is not intended to be exhaustive or to limit the implementations to the precise forms disclosed. Modifications may be made in light of the above disclosure or may be acquired from practice of the implementations. For example, various aspects of this disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

A computer program product embodiment (“CPP embodiment” or “CPP”) is a term used in this disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, RAM, ROM, erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc), or any suitable combination of the foregoing. A computer-readable storage medium, as that term is used in this disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

As used herein, the term “component” or “module” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

When “a processor” or “one or more processors” (or another device or component, such as “a controller” or “one or more controllers”) is described or claimed (within a single claim or across multiple claims) as performing multiple operations or being configured to perform multiple operations, this language is intended to broadly cover a variety of processor architectures and environments. For example, unless explicitly claimed otherwise (e.g., via the use of “first processor” and “second processor” or other language that differentiates processors in the claims), this language is intended to cover a single processor performing or being configured to perform all of the operations, a group of processors collectively performing or being configured to perform all of the operations, a first processor performing or being configured to perform a first operation and a second processor performing or being configured to perform a second operation, or any combination of processors performing or being configured to perform the operations. For example, when a claim has the form “one or more processors configured to: perform X; perform Y; and perform Z,” that claim should be interpreted to mean “one or more processors configured to perform X; one or more (possibly different) processors configured to perform Y; and one or more (also possibly different) processors configured to perform Z.”

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method comprising:
receiving input data that includes a satellite observation of a farm field, weather forecasts for the farm field, and geo-coordinates of the farm field;
determining a target soil moisture level that is based on tunneling activity of earthworms in the farm field; and
outputting the target soil moisture level.

2. The method of claim 1, wherein the input data includes an excess soil nutrient level in the farm field.

3. The method of claim 1, further comprising:
estimating an excess soil nutrient level in the farm field based on the satellite observation, wherein the determining of the target soil moisture level comprises determining the target soil moisture level based on the excess soil nutrient level.

4. The method of claim 1, further comprising:
predicting rainfall and temperature patterns that affect the tunneling activity of the earthworms based on the weather forecasts, wherein the determining of the target soil moisture level comprises determining the target soil moisture level based on the rainfall and temperature patterns.

5. The method of claim 1, wherein the input data includes digital elevation data for the farm field, wherein the method comprises determining areas of the farm field with high potential for water runoff based on the digital elevation data and the geo-coordinates, and wherein the determining of the target soil moisture level comprises determining the target soil moisture level based on the areas with high potential for water runoff.

6. The method of claim 1, wherein the determining of the target soil moisture level comprises determining the target soil moisture level based on a crop nutrient requirement and a crop water stress constraint.

7. The method of claim 1, wherein the determining of the target soil moisture level comprises determining the target soil moisture level based on minimizing greenhouse gas emissions from the earthworms.

8. The method of claim 7, wherein the determining of the target soil moisture comprises determining the target soil moisture using a cost function that is based on the tunneling activity of the earthworms, the greenhouse gas emissions from the earthworms, and crop nutrient requirements.

9. The method of claim 8, wherein the determining of the target soil moisture level comprises determining the target soil moisture level based on a soil type, a soil texture, and a soil organic matter content of the farm field.

10. A computer system comprising:
a processor set;
one or more computer-readable storage media; and
program instructions stored on the one or more computer-readable storage media to cause the processor set to perform operations comprising:
receiving input data comprising satellite observations, weather forecasts, farm geo-coordinates, digital elevation data, and excess soil nutrient levels associated with a farm field;
determining, based on the input data, a target soil moisture level associated with accelerating tunneling activity of earthworms while minimizing greenhouse gas emissions from the earthworms; and
outputting the target soil moisture level.

11. The computer system of claim 10, further comprising:
identifying waterbodies with high potential for algal bloom and agricultural runoff based on the satellite observations and image analysis.

12. The computer system of claim 10, wherein the outputting of the target soil moisture level comprises generating guidance information associated with an optimal soil moisture level for the target soil moisture level and an earthworm deployment strategy to prevent nutrient runoff and control algal blooms.

13. The computer system of claim 10, wherein the operations further comprise:
determining a proximity of the farm field and one or more surrounding farm fields to one or more waterbodies; and
determining how much the farm field and the surrounding farm fields contribute to runoff water with excess soil nutrients based on the proximity and a digital elevation of the farm field.

14. The computer system of claim 10, wherein the determining of the target soil moisture level comprises determining the target soil moisture level based on soil type, soil texture, and soil organic matter content.

15. A computer program product comprising:
one or more computer-readable storage media; and
program instructions stored on the one or more computer-readable storage media to perform operations comprising:
detecting one or more waterbodies having high potential for algal bloom using a combination of satellite observations, weather forecast, farm geo-coordinates, and digital elevation information;
identifying a farm field having a high potential for contributing to water runoff with excess soil nutrients, based on the one or more waterbodies, the satellite observations, the weather forecast, and the digital elevation information;
estimating an excess soil nutrient level for the farm field; and
outputting an optimal number of earthworms to deploy in the farm field based on the estimated excess soil nutrient level.

16. The computer program product of claim 15, wherein the instructions further comprise:
outputting a target soil moisture level for the farm field based on the excess soil nutrient level, the one or more waterbodies, the satellite observations, the weather forecast, and the digital elevation information.

17. The computer program product of claim 15, wherein the instructions further comprise:
predicting, using a machine learning model, an optimal target soil moisture level based on historical data and real-time sensor data.

18. The computer program product of claim 15, wherein the instructions further comprise:
determining a target soil moisture level based on the estimated excess soil nutrient level, a soil type, a soil texture, and a soil organic matter content of the farm field.

19. The computer program product of claim 15, wherein the outputting of the number of earthworms comprises outputting the number of earthworms for the farm field based on an estimate of greenhouse gas emissions emitted by the number of earthworms to deploy and existing earthworms in the farm field.

20. The computer program product of claim 15, wherein the instructions further comprise:
outputting a target soil moisture level that is based on earthworm survival constraints.
